(19) United States
(12) Patent Application Publication
(10) Pub. No.: US 2006/0084800 A1
(43) Pub. Date: Apr. 20, 2006

(54) Synthesis of aldonolactones, aldarolactones, and aldarodilactones using azeotropic distillation

(76) Inventor: Henry Keith Chenault, Hockessin, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/173,462

(22) Filed: Jun. 30, 2005

Related U.S. Application Data
(60) Provisional application No. 60/584,832, filed on Jun. 30, 2004.

(51) Int. Cl. C07D 309/30; C07H 3/00

(52) U.S. Cl. 536/124; 549/292

(57) ABSTRACT

Processes for making lactones and dilactones from aldaric acids, aldonic acids, and their corresponding salts by dehydrative cyclization and azeotropic distillation. The processes can be carried out in the presence of water because water is removed by azeotropic distillation.

### FIELD OF INVENTION

This invention is directed to processes for producing lactones or dilactones from aldonic acids, aldaric acids or aldarolactones, or salts thereof. The processes include dehydratively cyclizing a reaction mixture comprising a 5- to 8-carbon aldonic acid, 5- to 8-carbon aldaric acid or 5- to 8-carbon aldarolactone, or mixture thereof, in a solvent mixture, and removing water by azeotropic distillation.

### BACKGROUND

Lactones and dilactones derived ultimately from renewable carbohydrate resources are highly functionalized monomers that are useful as synthetic intermediates, chiral starting materials, enzyme inhibitors, and monomers for polymer synthesis.

Aldaric acids and aldonic acids are oxidized derivatives of aldose carbohydrates. When only the aldehyde of an aldose is oxidized, an aldonic acid is formed. If both the aldehyde and terminal alcohol of an aldose are oxidized, an aldaric acid is formed. Lactones and dilactones can be produced from these acids via dehydrative cyclization, typically by heating the parent aldonic or aldaric acid under vacuum (Hirasaka, Y.; Umemoto, K. Chem. Pharm. Bull. 1965, 13, 325-329). Recent publications and patents demonstrate that this technology has not changed for many years (U.S. Pat. No. 6,049,004). Even with heating under vacuum, conversion to the desired lactone is often incomplete (Conchie, J.; Hay, A. J.; Strachan, I.; Levvy, G. A. Biochem. J. 1967, 102, 929-941), requiring purification of the desired lactone by recrystallization (Isbell, H. S.; Frush, H. L. Bur. Standards J. Research 1933, 11, 649-664) or column chromatography. Furthermore, heating under vacuum often generates impurities due to thermal decomposition.

Hashimoto, et al. (Hashimoto, K.; et al., Makromol. Chem., Rapid Commun. 1990, 11, 393-396) disclose the synthesis of D-glucaro-1,4:6,3-dilactone by repeated lyophilization of glucaric acid from dioxane.

Although synthesis of an aldonolactone using an alcohol to effect azeotropic removal of water has been described (U.S. Pat. No. 1,830,618), the method suffers from the formation esters as by-products. While known processes may be acceptable for generating grams to tens of grams of material, they can be impractical for preparing tens to thousands of pounds of material. High vacuum, long residence time, and the high substrate surface area required by the solvent-free method are all impediments to practicing these methods on large scale.

What is needed, therefore, is a process that can be effectively carried out on a larger scale than previously reported methods, and that will also generate lower quantities of decomposition by-products.

### SUMMARY OF THE INVENTION

The present invention provides processes for preparing lactones or dilactones comprising the dehydrative cyclization of a reaction mixture comprising a 5- to 8-carbon aldonic acid, 5- to 8-carbon aldaric acid or 5- to 8-carbon aldarolactone, or mixture thereof, in a solvent mixture comprising one or more suitable solvents, wherein water is removed by azeotropic distillation.

One aspect of the present invention is a process for preparing a lactone or dilactone comprising:
a) providing a reaction mixture comprising:
   i) a solvent mixture comprising about 0 to about 50 volume % of water and about 100 to about 50 volume % of a suitable solvent, based on the total volume of the solvent mixture; and
   ii) a starting material comprising one or more compounds selected from 5- to 8-carbon aldonic acids, 5- to 8-carbon aldaric acids, and 5- to 8-carbon aldarolactones; and
b) heating the reaction mixture to effect dehydrative cyclization of the compound in the starting material and removal of water by azeotropic distillation.

In some embodiments, the suitable solvent comprises an ether, ketone, or ester having a boiling point of about 80 to about 150° C., that forms an azeotrope with water, the azeotrope having a boiling point below that of water and below that of the suitable solvent. Preferably the suitable solvent has a boiling point of about 100 to about 120° C. In preferred embodiments, the solvent is methyl ethyl ketone, methyl isobutyl ketone, 3-pentanone, cyclopentanone, dioxane, ethylene glycol diethyl ether or propyl acetate. Also preferably, the lactone or dilactone is soluble in the suitable solvent above about 25° C. and precipitates at or below 25° C. The solvent mixture can further comprise water or acetone.

In some embodiments, the reaction mixture comprises an equilibrium mixture of an aldaric acid and one or more of the corresponding aldarolactone or aldarodilactone, or an equilibrium mixture of an aldonic acid and the corresponding aldonolactone. In some embodiments the aldaric acid is glucaric acid. In some embodiments, the aldonic acid is gluconic acid.

In some embodiments, the aldonic acid, aldaric acid or aldarolactone contains one or more protected hydroxyl groups. The hydroxyl groups can be protected as ethers, acetals, carboxylic esters, or sulfonate esters.

In some embodiments, the 5- to 8-carbon aldonic acid, 5- to 8-carbon aldaric acid or 5- to 8-carbon aldarolactone is D, L, racemic or a nonracemic mixture in its enantiomeric configuration. The reaction mixture can also comprise an aldaric acid that has a plane of symmetry and thus exists in only a meso configuration.

In some embodiments, the aldonic acid, aldaric acid or aldarolactone is generated in situ from the corresponding Group I, Group II, or ammonium salt, or mixture thereof by acidification. The salt can be a sodium, potassium, lithium, cesium, magnesium, calcium, or ammonium salt, and the acid can be sulfuric acid, HCl, phosphoric acid, HF, oxalic acid, trifluoroacetic acid, or an acidic cation exchange resin. Optionally any precipitate formed during the generation of the aldonic acid, aldaric acid or aldarolactone in situ can be removed.

### DETAILED DESCRIPTION

The present invention provides processes for the preparation of a lactone or dilactone by dehydrative cyclization of a 5- to 8-carbon aldonic acid, 5- to 8-carbon aldaric acid or 5- to 8-carbon aldarolactone, or mixture thereof, in a solvent mixture, where the solvent mixture comprises one or more of a suitable solvent, wherein water is removed by azeotropic distillation.

The reaction mixture can comprise, for example, gluconic, mannonic, galactonic, idonic, allonic, altronic, gulonic, talonic, ribonic, xylonic, arabinonic, lyxonic, glucaric, mannaric, galactaric, idaric, allaric, altraric, ribaric, xylaric or arabinaric acid.

As used herein, an aldaric acid is a derivative of an aldose carbohydrate in which the terminal aldehyde and alcohol groups have been converted to carboxylic acids. An example of an aldaric acid is the aldaric acid derived from glucose, glucaric acid: HOOC—(CHOH)₄—COOH. Any aldaric acid that can form a lactone or dilactone is suitable for the instant invention, as described below. The aldaric acid can be in any enantiomeric form. Aldaric acid starting materials include but are not limited to glucaric (=gularic), mannaric, galactaric, idaric, allaric, altraric (=talaric), ribaric, xylaric, and arabinaric (=lyxaric) acids. Preferred are five to eight carbon aldaric acids; more preferred is glucaric acid; most preferred is D-glucaric acid.

Six-carbon aldaric acids that can form two cis-fused five-membered lactones (γ-lactones) do so and thus generate dilactone products. The other six-carbon aldaric acids and the five-carbon aldaric acids form monolactones as their ultimate lactonization products.

Pictured below are the ultimate products formed when six- and five-carbon aldaric acids are dehydratively lactonized. In cases where the starting material is optically active, only one enantiomeric product is pictured. It is understood that the other enantiomeric starting material would form the enantiomeric product (e.g., L-mannaric acid would give L-mannaro-1,4:6,3-lactone) and that mixtures of stereoisomers, including racemates, would form corresponding mixtures of stereoisomeric products. It is also understood that various salts of the aldaric acids may be converted into the free acid in situ and then lactonized.

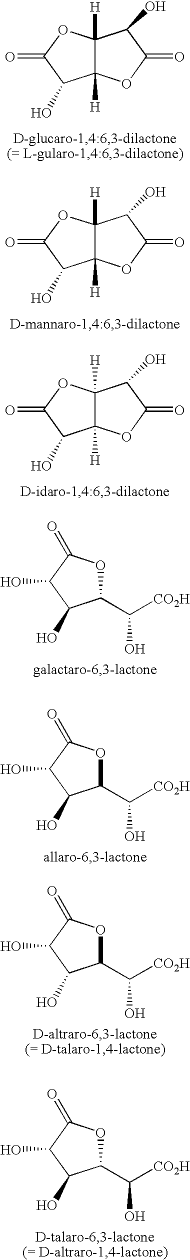

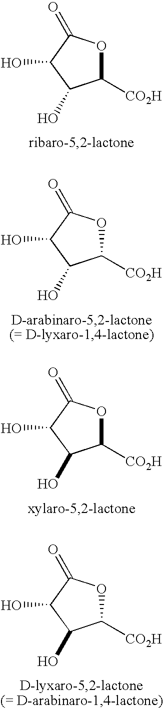

Because the molecules have carboxyl groups at both ends, there is potential for numbering from either end (e.g., D-glucaric acid has the same absolute structure as L-gularic acid, and D-altraro-6,3-lactone has the same absolute structure as D-talaro-1,4-lactone).

D-Glucaric acid (CAS Reg. No. 87-73-0, =L-gularic acid) gives D-glucaro-1,4:6,3-dilactone (CAS Reg. No. 826-91-5, =L-gularo-1,4:6,3-dilactone). L-Glucaric acid (CAS Reg. No. 5627-26-9, =D-gularic acid) gives L-glucaro-1,4:6,3-dilactone (=D-gularo-1,4:6,3-dilactone).

D-Mannaric acid (CAS Reg. No. 22076-54-60) gives D-mannaro-1,4:6,3-dilactone (CAS Reg. No. 2900-01-8). L-Mannaric acid gives L-mannaro1,4:6,3-dilactone (CAS Reg. No. 214038-58-1, although this CAS registry number is incorrectly named L-mannonic acid di-γ-lactone).

D-Idaric acid (CAS Reg. No. 33012-63-4) gives D-idaro-1,4:6,3-dilactone. L-Idaric acid (CAS Reg. No. 80876-58-0) gives L-idaro-1,4:6,3-dilactone.

Galactaric acid (CAS Reg. No. 526-99-8, meso and thus optically inactive) gives (racemic) DL-galactaro-6,3-dilactone (=DL-galactaro-1,4-dilactone).

Allaric acid (CAS Reg. No. 527-00-4, meso and thus optically inactive) gives (racemic) DL-allaro-6,3-dilactone (=DL-allaro-1,4-dilactone).

D-Altraric acid (CAS Reg. No. 117468-78-7, =D-talaric acid) gives a mixture of D-altraro-1,4-lactone (CAS Reg. No. 91547-68-1, =D-talaro-6,3-lactone, although incorrectly named in CAS registry as D-talomucic acid 1,4-lactone) and D-altraro-6,3-lactone (CAS Reg. No. 91547-67-0, =D-talaro-1,4-lactone, although incorrectly named in CAS registry as D-talomucic acid 6,3-lactone). L-Altraric acid (CAS Reg. No.117468-79-8, =L-talaric acid) gives a mixture of L-altraro-1,4-lactone (=L-talaro-6,3-lactone) and L-altraro-6,3-lactone (=L-talaro-1,4-lactone).

Ribaric acid (meso, CAS Reg. No. 33012-62-3) gives (racemic) DL-ribaro-5,2-lactone (CAS Reg. No. 85114-92-7, DL-ribaro-1,4-lactone).

D-Arabinaric acid (CAS Reg. No. 20869-04-9, =D-lyxaric acid) gives a mixture of D-arabinaro-1,4-lactone (=D-lyxaro-5,2-lactone) and D-arabinaro-5,2-lactone (=D-lyxaro-1,4-lactone). L-Arabinaric acid (CAS Reg. No. 608-54-8, =D-lyxaric acid) gives a mixture of L-arabinaro-1,4-lactone (=L-lyxaro-5,2-lactone) and L-arabinaro-5,2-lactone (=L-lyxaro-1,4-lactone).

Xylaric acid (meso, CAS Reg. No.10158-64-2) gives (racemic) DL-xylaro-5,2-lactone (=DL-xylaro-1,4-lactone).

An aldonic acid, as used herein, is a derivative of an aldose carbohydrate in which the terminal aldehyde group has been converted to a carboxylic acid. An example of an aldonic acid is the aldonic acid derived from glucose, gluconic acid: HOOC—(CHOH)₄—CH₂OH. Any aldonic acid that can form a lactone is suitable for the instant invention, as described below. The aldonic acid can be in any enantiomeric form.

Suitable aldonic acids include, but are not limited to, gluconic, mannonic, galactonic, idonic, allonic, altronic, gulonic, talonic, ribonic, xylonic, arabinonic, and lyxonic acids. Preferred are 5-8 carbon acids; most preferred is gluconic acid.

Pictured below are the 12 1,4-lactones (γ-lactones) formed by the 8 six-carbon and 4 five-carbon aldonic acids. Because aldonic acids have only one carboxyl group, they can form only one lactone ring. Some of the products shown below will be formed in the presence of their corresponding 1,5-lactone (6-lactone), but the 1,4-lactone is usually the major product, especially at higher temperatures.

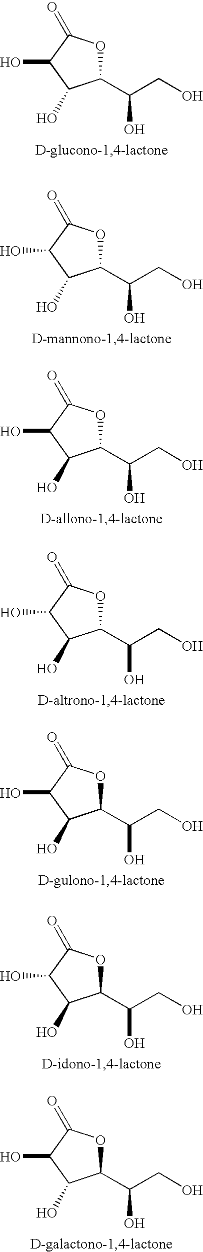

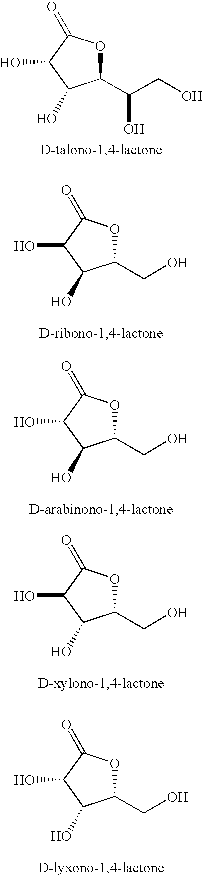

As with the aldarolactones above, only one enantiomeric form of each aldonolactone is pictured. One skilled in the art will recognize that the other enantiomeric starting material will give the enantiomeric product and that mixtures of stereoisomers, including racemates, will form corresponding mixtures of stereoisomeric products. Salts of the aldonic acids can be converted into the free acid in situ and then lactonized.

D-Gluconic acid (CAS Reg. No. 526-95-4) gives D-glucono-1,4-lactone (1198-69-2). L-Gluconic acid (CAS Reg. No. 157663-13-3) gives L-glucono-1,4-lactone (CAS Reg. No. 74464-44-1).

D-Mannonic acid (CAS Reg. No. 642-99-9) gives D-mannono-1,4-lactone (CAS Reg. No. 26301-79-1). L-Mannonic acid (CAS Reg. No. 51547-37-6) gives L-mannono-1,4-lactone (CAS Reg. No. 22430-23-5).

D-Allonic acid (CAS Reg. No. 21675-42-3) gives D-allono-1,4-lactone (CAS Reg. No. 29474-78-0). L-Allonic acid gives L-allono-1,4-lactone (CAS Reg. No. 78184-43-7).

D-Altronic acid (CAS Reg. No. 22430-69-9) gives D-altrono-1,4-lactone (CAS Reg. No. 83602-36-2). L-Altronic acid gives L-altrono-1,4-lactone (CAS Reg. No. 119008-75-2).

D-Gulonic acid (CAS Reg. No. 20246-33-7, or CAS Reg. No. 66905-24-6 for the monohydrate) gives D-gulono-1,4-lactone (CAS Reg. No. 6322-07-2). L-Gulonic acid (CAS Reg. No. 526-97-6) gives L-gulono-1,4-lactone (CAS Reg. No.1128-24-1).

D-ldonic acid (CAS Reg. No. 488-33-5) gives D-idono-1,4-lactone (CAS Reg. No. 161168-87-2). L-Idonic acid (CAS Reg. No. 1114-17-6) gives L-idono-1,4-lactone (CAS Reg. No. 1128-24-1).

D-Galactonic acid (CAS Reg. No. 576-36-3) gives D-galactono-1,4-lactone (CAS Reg. No. 2782-07-2). L-Galactonic acid (CAS Reg. No. 28278-17-3) gives L-galactono-1,4-lactone (CAS Reg. No. 1668-08-2).

D-Talonic acid (CAS Reg. No. 20246-35-9) gives D-talono-1,4-lactone (CAS Reg. No. 23666-11-7). L-Talonic acid gives L-talono-1,4-lactone (CAS Reg. No. 127997-10-8).

D-Ribonic acid (CAS Reg. No. 642-98-8) gives D-ribono-1,4-lactone (CAS Reg. No. 5336-08-3). L-Ribonic acid gives L-ribono-1,4-lactone (CAS Reg. No.133908-85-7).

D-Arabinonic acid (CAS Reg. No. 488-30-2) gives D-arabinono-1,4-lactone (CAS Reg. No. 2782-09-4). L-Arabinonic acid (CAS Reg. No. 608-53-7) gives L-arabinono-1,4-lactone (CAS Reg. No. 51532-86-6).

D-Xylonic acid (CAS Reg. No. 526-91-0) gives D-xylono-1,4-lactone (CAS Reg. No.15384-37-9). L-Xylonic acid (CAS Reg. No. 4172-44-5) gives L-xylono-1,4-lactone (CAS Reg. No. 68035-75-6).

D-Lyxonic acid (CAS Reg. No. 526-92-1) gives D-lyxono-1,4-lactone (CAS Reg. No. 15384-34-6). L-Lyxonic acid (CAS Reg. No. 4172-43-4) gives L-lyxono-1,4-lactone (CAS Reg. No.104196-15-8).

The starting reactants can contain one or more hydroxyl groups that have been modified to give either a “deoxy” or a protected derivative. By “protected” is meant blocking the reactivity of a hydroxyl group with one or more reagents while a chemical reaction is carried out at an alternative reactive site of the same compound. Protecting groups are well known in the art and any suitable group can be used. Useful hydroxyl protecting groups include ethers, acetals, and carboxylic or sulfonate esters.

Since many aldonic and aldaric acids exist in solution in equilibrium with their lactone and (if possible) dilactone derivatives, the starting material may be an equilibrium mixture of an aldonic or aldaric acid and its various lactone and (if possible) dilactone derivatives. Furthermore, since aldonic and aldaric acids generally exist in both D and L enantiomeric configurations, the starting material may be D, L, racemic (DL), or an unequal mixture of enantiomers. Some aldaric acids have a plane of symmetry and thus exist in only a meso configuration.

The starting aldonic or aldaric acid or corresponding lactone may be generated by acidifying a Group I, Group II, or ammonium salt precursor of the parent acid or monolactone. Salts that may serve as precursors include but are not limited to sodium, potassium, lithium, cesium, magnesium, calcium, and ammonium salts. A mixture of salt forms having different cations may also be used as a precursor to form the aldonic or aldaric acid. Acids useful for generating aldonic and aldaric acids by acidifying precursor salts include strong mineral acids, carboxylic acids, or polymer bound acids, such as but not limited to sulfuric, hydrochloric, phosphoric, hydrofluoric, oxalic, and trifluoroacetic acids, hydrogen chloride, hydrogen fluoride, and polymeric or solid-phase acids (e.g., strongly acidic cation exchange resins). The starting aldonic or aldaric acid can be generated in solution in water, in a suitable organic solvent such as acetone, or in a mixture of said solvent and water. Any precipitate formed may optionally be removed by any means, such as filtration, before proceeding.

The starting material may optionally be a mixture of different aldonic and/or aldaric acids having different numbers of carbon atoms, different diastereomeric configurations, and/or different numbers of carboxylic acid groups. The mixtures can also be generated in whole or in part by acidifying the appropriate precursor salts.

In some embodiments, the starting material can be a mixture of one or more of an aldonic acid, an aldaric acid, an aldonolactone, an aldarolactone, and an aldarodilactone. The mixture can be an equilibrium mixture of an aldaric acid or an aldonic acid with its corresponding aldarolactone, aldonolactone, and/or its corresponding aldarodilactone if one exists. Preferably, the aldonic acids, aldaric acids, aldonolactones, aldarolactones and aldarodilactones contain from 5 to 8 carbon atoms.

In a process of the present invention, the starting materials are combined with a suitable solvent. The starting materials can be first dissolved in water, acetone, or a water-acetone mixture before combining with the suitable solvent. The amount of starting material dissolved in the suitable solvent is not critical, and is limited primarily by the quantity of material that will dissolve in the solvent. While the concentration at which the process is run is limited only by the solubility of the starting material, the process is preferably run at about 1 to about 50 weight % solids loading. That is, the starting material is typically dissolved initially in about 1 to about 99 weight equivalents of solvent. More preferably, the process is run at about 10 to about 45 weight % solids loading. That is, the substrate is dissolved initially in about 1.2 to about 9 weight equivalents of solvent.

The combined mixture is then heated, thereby promoting the formation of a lactone or dilactone by dehydrative cyclization, and azeotropically distilling the combined mixture, to remove water.

As used herein, “suitable solvent” means any solvent or mixture of solvents that is substantially inert to all reagents and products, dissolves the starting materials, and forms an azeotrope with water that has a boiling point below that of water and below that of the suitable solvent. Suitable solvents include ethers, ketones, and esters, such as but not limited to methyl ethyl ketone, methyl isobutyl ketone, 3-pentanone, cyclopentanone, dioxane, ethylene glycol diethyl ether and propyl acetate. The suitable solvent can also further comprise water or acetone. Preferred solvents have a boiling point about 80 to 150° C., more preferred about 90 to 130° C.; and even more preferred about 100 to 120° C. Solvents with alcoholic functionalities, such as butanol, ethanol, cyclohexanol and phenol, are generally not preferred, as they can lead to the formation of aldonic or aldaric acid esters. For ease in separation, the product is preferably soluble in the suitable solvent when the solvent is hot but precipitates when the solvent is cooled to −30 to 25° C., allowing the product to be collected by filtration, centrifugation, or other physical separation processes.

It is believed that the choice of solvent or the temperature at which lactonization is conducted may affect the product distribution and thus may favor one particular regioisomeric lactone over another, either kinetically or thermodynamically. For example, aldonic and aldaric acids often can form either five-membered (γ) or six-membered (δ) ring lactones. Talaric acid (also known as altraric) can form either the 1,4- or 6,3-lactone, and arabinaric acid (also known as lyxaric acid) can form either the 1,4- or 5,2-lactone. It is not intended that the processes of the present invention be limited to the formation of any particular enantiomer or mixture thereof.

The processes disclosed herein are useful for converting glucaric acid or glucarolactone into glucaro-1,4:6,3-dilactone, mannaric acid or mannarolactone into mannaro-1,4:6,3-dilactone, and idaric acid or idarolactone into idaro-1,4:6,3-dilactone. Other 5 and 6-carbon aldonic and aldaric acids form monolactone products.

### EXAMPLES

The following materials are used in the Examples:
Calcium D-glucarate tetrahydrate (D-saccharic acid, calcium salt),
Spectrum Chemicals, 1001, FW 320.27
Sulfuric Acid, reagent grade, 95-98%, FW 98.07, d 1.84
Acetone, reagent grade, 99.5+%
Methyl isobutyl ketone (MiBK, 4-methyl-2-pentanone), reagent grade, 99+%

### Example 1

Sulfuric acid (312.5 g, 3.122 moles) was added over a period of 30 minutes to a stirred suspension of calcium D-glucarate tetrahydrate (1000 g, 3.122 moles) in 3.1 L of 97.5:2.5 acetone-water (prepared by mixing 3044 mL of acetone with 78 mL of water).

The stirred mixture was heated at reflux for 4 hours, allowed to cool to room temperature (20-25° C.), stirred at room temperature for 1-2 hours, and then filtered with suction to remove the precipitated calcium sulfate. At no time did the reaction become homogeneous. The precipitate was washed three times with 1.0 L of 97.5:2.5 acetone-water, each time suspending the precipitate in the solvent and then sucking the solvent through.

Since some of the acetone was lost by evaporation during the filtration process, the filtrate and washings were combined and adjusted back up to 6.2 L by addition of acetone, typically about 1.6 L. MiBK (7.75 L) was added to the aqueous acetone solution, and the vigorously stirred solution was heated so as to remove the acetone by fractional distillation. Thus, 6.2 L of acetone containing some water and some MiBK was distilled off (pot temp. 65-95° C., still head temp. 56-85° C.). Distillation was continued until the pot temperature reached 115-119° C. At this point, distillation was discontinued and the reaction was heated at reflux for 30 minutes. After 30 minutes at reflux, distillation was resumed until a total of 8.1 L had been removed from the original reaction volume.

The reaction mixture was filtered hot to separate the solution from about 30 g of a brown oil that adhered to the surface of the glass reaction vessel. The reaction filtrate was allowed to cool with vigorous stirring under a blanket of dry nitrogen. The solution was seeded with 0.5-0.6 g of GDL (D-glucaro-1,4:6,3-dilactone) and cooled to room temperature. Once the mixture had reached room temperature, crystallization was allowed to continue for 2-3 hours or overnight.

The white, crystalline GDL was collected by filtration, rinsed with one 750-mL portion of MiBK, dried under a stream of nitrogen and then in vacuo. Yield was 250-270 g (46-50%).

The mother liquor from the first crystallization (about 4.7 L) was further concentrated to 1.9 L by distillation. The concentrated mother liquor was filtered hot, cooled with vigorous stirring under a blanket of dry nitrogen as before, and seeded with 0.3 g of GDL. Once the mixture had reached room temperature, crystallization was allowed to continue for 2-3 hours or overnight.

The white, crystalline GDL was collected by filtration, rinsed with one 375-mL portion of MiBK, dried under a stream of nitrogen and then in vacuo. Yield was 125 g (23%).

Analysis was performed by 1H NMR and by GC (silylation with BSTFA-TMSCI, J&W DB-17 MS 30 m×0.32 mm×0.25 m column, oven temperature 120 -300° C.).

### Example 2

D-Gluconic acid (20 g of a 50 wt % solution in water) and 100 mL of cyclopentanone were combined and heated until a total of 22.5 mL of solvent had been removed by distillation. The reaction mixture was filtered hot, and the filtrate was allowed to begin cooling under an atmosphere of dry nitrogen. The solution was seeded with 5 mg of D-gluconolactone and allowed to sit overnight. The white, crystalline D-gluconolactone was collected by filtration, rinsed with 3 10-mL portions of MiBK, and dried under vacuum. Yield 3.1 g (34%) of what was by ¹H and ¹³C NMR a 2:1 mixture of D-glucono-1,4-lactone and D-glucono-1,5-lactone. More product was collected and was shown by ¹H and ¹³C NMR to be a 3:2 mixture of D-glucono-1,4-lactone and D-glucono-1,5-lactone.

### Example 3

A 50-gallon reactor was charged with 113 lb of acetone and 48.5 lb of calcium D-glucarate tetrahydrate over a period of 1 h, the charge port and funnel being rinsed through to the reactor with 4.0 lb of Dl water. Sulfuric acid (15.2 lb) was charged to a stainless steel bomb and pumped from there into the reactor over a period of 1 hour, during which time the pot temperature rose from 22.8 to 27.8° C. The bomb and transfer lines were rinsed through to the reactor with 3.5 lb of Dl water. The mixture was stirred overnight (19 h) at 50 rpm, at ambient temperature, under nitrogen.

The mixture was then filtered through a sparkler filter dressed with duck cloth and 40-μm Dacron® cloth to give 81.5 lb of filtrate. The kettle and filter cake were rinsed through with a mixture of 109.5 lb of acetone and 7.2 lb of Dl water, divided into three portions. The combined filtrate and washings (209.5 lb) were adjusted to 275 lb by addition of 65.5 lb of acetone and stored in a 55-gallon polylined drum.

The cleaned 50-gallon reactor was than charged with exactly half (137.5 lb) of the product solution from above and 131 lb of MiBK (methyl isobutyl ketone) over a period of 32 min. The mixture was stirred at 50 rpm and heated to reflux over the next 2 hours. Over the next 7 hours, 175.5 lb of acetone/water/MiBK were distilled off.

The contents of the 50-gallon reactor were transferred through a line heated at 80° C. and a 200-μm in-line filter to a 20-gallon kettle, which was cooled to 40° C. and then 32° C. About 50 mL of the solution was removed, seeded with crystals of GDL to initiate crystallization, and then returned to the 20-gallon reactor to initiate crystallization of the product.

After stirring gently overnight, the material was transferred to a sparkler filter, and 14.5 lb of MiBK were used to rinse out the reactor and rinse through the filter cake. The filter cake (12 lb) was dried in a vacuum oven at 50° C. with a slight nitrogen purge for about a day and a half to give 3.244 kg of crystalline GDL (27.1% yield), purity 99.4% by ¹H NMR and 99.6% by GC.

The second half of the product solution (137.3 lb) was treated as above, except that only 170.0 lb of acetone/water/MiBK were removed. Dried GDL weighed 2.248 kg (18.8% yield) and was 99.7% pure by ¹H NMR and GC.

The combined mother liquors and MiBK rinses were returned to the 50-gallon reactor, stirred at 50 rpm and heated to reflux over the next 4 hours. Over the next 4.5 hours, 100.0 lb of solvent were distilled off.

The contents of the 50-gallon reactor were transferred to the 20-gallon kettle as above. An aliquot was removed, seeded, and returned to the mixture at 42° C.

After the slurry had stirred overnight, the material was transferred to a sparkler filter, and 17.5 lb of MiBK were used to rinse out the reactor and rinse through the filter cake. The filter cake was rinsed with an additional 7.0 lb of MiBK and dried in a vacuum oven to give 1.879 kg (15.6% yield) of GDL that was 99.5% pure by 1H NMR and 99.8% pure by GC.

What is claimed is:

1. A process for preparing a lactone or dilactone comprising:
a) providing a reaction mixture comprising:
i) a solvent mixture comprising about 0 to about 50 volume % of water and about 100 to about 50 volume % of a suitable solvent, based on the total volume of the solvent mixture; and
ii) a starting material comprising one or more compounds selected from 5- to 8-carbon aldonic acids, 5- to 8-carbon aldaric acids, and 5- to 8-carbon aldarolactones; and
b) heating the reaction mixture to effect dehydrative cyclization of the compound in the starting material and removal of water by azeotropic distillation.

2. The process of claim 1 wherein the solvent mixture comprises about 1 to about 50 volume % of water and about 99 to 50 volume % of a suitable solvent.

3. The process of claim 1 wherein the suitable solvent comprises an ether, ketone, or ester having a boiling point of 80 to 150° C. that forms an azeotrope with water with a boiling point below that of water and below that of the suitable solvent.

4. The process of claim 3 wherein the suitable solvent has a boiling point of 100 to 120° C.

5. The process of claim 1 wherein the lactone or dilactone is soluble in the suitable solvent above 25° C. and precipitates at or below 25° C.

6. The process of claim 3 wherein the suitable solvent is methyl ethyl ketone, methyl isobutyl ketone, 3-pentanone, cyclopentanone, dioxane, ethylene glycol diethyl ether or propyl acetate.

7. The process of claim 1 wherein the solvent mixture comprises at least one of water and acetone.

8. The process of claim 1 wherein the reaction mixture comprises an equilibrium mixture of an aldaric acid and one or more of the corresponding aldarolactone or aldarodilactone, or an equilibrium mixture of an aldonic acid and the corresponding aldonolactone.

9. The process of claim 1 wherein the reaction mixture comprises one or more acid selected from: gluconic, mannonic, galactonic, idonic, allonic, altronic, gulonic, talonic, ribonic, xylonic, arabinonic, lyxonic, glucaric, mannaric, galactaric, idaric, allaric, altraric, ribaric, xylaric and arabinaric acids.

10. The process of claim 9 wherein the aldaric acid is glucaric acid or where the aldonic acid is gluconic acid.

11. The process of claim 1 wherein the aldonic acid, aldaric acid or aldarolactone contains one or more protected hydroxyl groups.

12. The process of claim 11 wherein the hydroxyl groups are protected as ethers, acetals, carboxylic esters, or sulfonate esters.

13. The process of claim 1 wherein the aldonic acid, aldaric acid or aldarolactone is D, L, racemic or a nonracemic mixture in its enantiomeric configuration.

14. The process of claim 1 wherein the reaction mixture comprises an aldaric acid that has a plane of symmetry and thus exists in only a meso configuration.

15. The process of claim 1 wherein the aldonic acid, aldaric acid or aldarolactone is generated in situ from the corresponding Group I, Group II, or ammonium salt, or mixture thereof.

16. The process of claim 15 wherein the salt is a sodium, potassium, lithium, cesium, magnesium, calcium, or ammonium salt.

17. The process of claim 16 wherein the salt is calcium glucarate.

18. The process of claim 15 wherein the aldonic acid, aldaric acid or aldarolactone is generated in situ via the addition of sulfuric acid, hydrochloric acid, phosphoric acid, hydrofluoric acid, oxalic acid, trifluoroacetic acid, or an acidic cation exchange resin.

19. The process of claim 15 wherein any precipitate formed during the generation of the aldonic acid, aldaric acid or aldarolactone in situ is removed.

20. The process of claim 1 wherein the suitable solvent comprises an ether, ketone, or ester.

21. The process of claim 1 wherein the reaction mixture is heated at a temperature of 80 to 150° C.

22. The process of claim 1 wherein the reaction mixture is heated at a temperature of 100 to 120° C.

23. The process of claim 1 further comprising generating the aldonic acid, aldaric acid or aldarolactone from the corresponding Group I, Group II, or ammonium salt, or mixture thereof, and optionally removing any precipitate.

24. The process of claim 1 further comprising combining the aldonic acid, aldaric acid or aldarolactone with water, acetone, or a water and acetone mixture.

25. The process of claim 1 further comprising:
a) cooling the solvent mixture to below 25° C. until the lactone or dilactone precipitates out of the solvent mixture;
b) separating the precipitated lactone or dilactone; and
c) optionally purifying the separated lactone or dilactone.
